(19) United States
(12) Patent Application Publication
(10) Pub. No.: US 2006/0057569 A1
(43) Pub. Date: Mar. 16, 2006

(54) Method for detecting hybridization events in nucleic acids

(76) Inventor: Christoph Charle, Berlin (DE)

Correspondence Address: Kevin C Hooper;Bryan Cave, 1290 Avenue of the Americas, New York, NY, 10104, US

(21) Appl. No.: 10/497,130

(22) PCT Filed: Nov. 25, 2002
(86) PCT No.: PCT/EP02/13215

§ 371 Date: Feb. 9, 2005

(30) Foreign Application Priority Data
Nov. 29, 2001 (DE) 101 58 516.0

(51) Int. Cl. C12Q 1/68; C07H 21/04

(52) U.S. Cl. 435/6; 536/25.32

(57) ABSTRACT

The invention relates to materials and methods for detecting the presence of and/or quantifying a target polynucleotide in a sample by using probe polynucleotides attached to the surface of an array, which probes are at least partially complementary to the target polynucleotide. A kit for carrying out the invention is also provided.

The invention relates, in particular, to a method for using solid phase-bound, labeled oligonucleotides, which are termed probe nucleic acids below, to detect RNA or DNA molecules (termed target nucleic acids below) in a sequence-specific manner.

Because of complementary regions in their base sequences, these probe nucleic acids, which can be DNA or DNA/PNA chimeras, are able to form intramolecular secondary structures which contain double-stranded regions. These double-stranded regions contain sequence motifs which can be recognized, and cleaved, by double strand-specific nucleases (restriction endonucleases) and which are here termed cleavage modules. The formation of the intramolecular cleavage modules within the probe nucleic acids is prevented by the probe nucleic acids hybridizing with complementary target nucleic acids in the sample to be investigated such that, following the digestion with double strand-specific nucleases, the hybridized probe nucleic acids can be distinguished from the unhybridized probe nucleases.

In a preferred embodiment, the method comprises specifically detecting different target nucleic acids, whose base sequences are not identical, using differently labeled probe nucleic acids which are all immobilized on a point or a defined area. The miniaturized arrangement of many such nucleic acids on a very small area is well known from biochip technology.

In customary methods for preparing DNA arrays, an unlabeled probe nucleic acid is applied, by means of in situ oligonucleotide synthesis [Fodor, S. P. A. et al., “Very large scale immobilized polymer synthesis” U.S. Pat. No. 5,424,186] or printing methods [Cheung, V. G. et al., “Making and reading microarrays”, Nature Genetics, vol. 21, January 1999; Bowtell, D. D. L., “Options available—from start to finish—for obtaining expression data by microarray”, Nature Genetics, vol. 21, 1999], to a solid matrix and covalently linked to this matrix. The probe nucleic acids are organized on the surface of the DNA array in the form of what are termed spots. Prior to a hybridization experiment, it is not possible to determine the quantity of nucleic acids which are immobilized on the solid matrix. Only by hybridizing the DNA array with the labeled sample nucleic acid and a second, labeled sample nucleic acid (double labeling, Wang, B., “Quantitative microarray hybridization assays”, U.S. Pat. No. 6,004,755), which serves as an internal standard, is it possible to compensate by calculation for the differences in the quantities of the unlabeled probe nucleic acids which are immobilized on the solid matrix.

Another method uses the principle of the nuclease protection test [Sambrook, J. et al., “Molecular Cloning” 2001, 3rd Edition, Cold Spring Harbor Laboratory] in order to break down unhybridized, that is single-stranded, labeled probe nucleic acids using single strand-specific nucleases [Kumar, R. et al., “Nuclease protection assays”, U.S. Pat. No. 5,770,370]. The precision of this method depends, in particular, on the stability of the duplexes composed of probe and target nucleic acids and on the specificity of the single strand-specific nucleases employed. The stability of a given nucleic acid duplex, which can be a DNA/DNA, DNA/RNA, DNA/PNA, RNA/RNA, RNA/PNA or PNA/PNA duplex, is specified by the number and strength of the Watson-Crick base pairings, between the complementary strands, which are mediated by hydrogen bonds [Lewin, B., “Genes VI”, 1997, Oxford University Press (and other current textbooks of molecular biology)]. In their end regions, nucleic acid duplexes are exposed to the influence of the surrounding medium, i.e. H₂O, which weakens the hydrogen bonds between the complementary strands. For this reason, the end regions of the duplexes are partially single-stranded under conditions which promote the reaction of single strand-specific nucleases (30-37° C.) and can be cleaved by the nucleases. Another disadvantage of this method is that single strand-specific nucleases such as S1 nuclease, mung bean nuclease, RNase A, RNase T1, exonuclease VII, Bal 31 nuclease, Micrococcus nuclease or nuclease Pi, do not cleave nucleic acids in a sequence-specific manner and very readily break down double-stranded regions if the ratio of the quantities of nucleic acid and nuclease in the reaction mixture is not precisely titered [Sambrook, J. et al., “Molecular Cloning” 2001, 3rd Edition, Cold Spring Harbor Laboratory].

Another system which can be used for detecting the hybridization of unlabeled sample nucleic acids uses what are termed molecular beacons [Tyagi, S. et al., “Detectably labeled dual conformation oligonucleotide probes, assays and kits”. U.S. Pat. No. 5,925,517] as probe nucleic acids which are bound covalently to glass microparticles or nanoparticles [Steemers, F. J. et al., “Screening unlabeled DNA targets with randomly ordered fiber-optic gene arrays”, 2000, Nature Biotech., vol. 18]. Molecular beacons are probe nucleic acids which can form intramolecular secondary structures and whose ends are linked covalently to different fluorophores which, as a consequence of the intramolecular secondary structure, are brought into close spatial proximity to each other. One of the two fluorophores (quencher) absorbs the photons which are emitted by the other fluorophore (emitter). The hybridization of a molecular beacon with a target nucleic acid dissociates the intramolecular secondary structure and the light which is emitted by the excited fluorophore (emitter) can no longer be absorbed by the quencher. This method has the crucial disadvantage that the signal/background ratio is approx. 25:1 [Steemers, F. J. et. al., “Screening unlabeled DNA targets with randomly ordered fiberoptic gene arrays”, 2000, Nature Biotech., vol. 18]. Since the differences in the transcription rate of different genes can be substantially larger than the signal/background ratio of molecular beacons, systems which operate on the basis of electron- or fluorescence resonance energy transfer are exclusively in the sphere of quantitatively determining target nucleic acids by means of amplifying a target sequence [Gelfand, D. H. et al.: “Detection of specific polymerase chain reaction product by utilizing the 5′ to 3′ exonuclease activity of Thermus aquaticus DNA-polymerase”, 1991. Proc. Natl. Acad. Sci., vol. 88 and U.S. Pat. No. 5,210,015 (1993); Tyagi, S. et al.: “Molecular Beacons: probes that fluoresce upon hybridization”, 1996, Nature Biotech., vol. 14] or in the context of determining nucleic acids in a nonquantitative manner [Steemers, F. J. et al., “Screening unlabeled DNA targets with randomly ordered fiber-optic gene arrays”, 2000, Nature Biotech., vol. 18].

At present, it is possible to immobilize more than 10 000 spots, that is more than 10 000 different nucleic acid probes, per cm² on DNA arrays [Bowtell, D. D. L., “Options available—from start to finish—for obtaining expression data by microarray”, Nature Genetics, vol. 21, January 1999]. The highest number of sample points (spots) which can be differentiated from each other is specified by the smallest spot size which it is technically possible to achieve. Since amounts of liquid in the nanoliter scale are transferred when preparing the array, this depends on physical quantities such as viscosity and surface tension of the transferred liquids. Another parameter which sets a lower limit to the size of the sample spots is the optical resolving power of the light microscope since all instruments for detecting fluorescence, luminescence or phosphorescence operate with optical systems which correspond to that of a light microscope (confocal laser scanning microscope). It is not possible, therefore, to fix an unlimited number of sample spots on a DNA array.

The present invention relates, therefore, to a method for detecting target nucleic acid by means of hybridization where, in the method,
a) a hybridization is carried out using at least one probe nucleic acid which is bound by one end to a solid phase. Each probe nucleic acid possesses a target nucleic acid sequence which is flanked, at the 3′ end, by a short nucleic acid sequence and, at the 5′ end, by a nucleic acid sequence which is complementary to it, which sequences are able to form a short DNA double strand. This double strand can be cleaved by a double strand-specific nuclease (restriction endonuclease) and thereby constitutes a cleavage module. In addition, the probe nucleic acid exhibits a label at the other end which is not bound to the solid phase;
b) at least one treatment with at least one double strand-specific nuclease is carried out. The digestion with the nuclease only cuts the cleavage modules which have formed a double strand. If it has not been possible to form a double strand because the target nucleic acid has hybridized with a nucleic acid to be detected, no double-stranded cleavage module is formed and the nucleic acid is not cut.
c) Finally, the amount of the label which is bound to the solid phase after step b) is determined.

In a preferred embodiment, several different probe nucleic acids, which contain different target sequences, are used in one methodological approach.

Preference is also given, in one method, to using several probe nucleic acids which possess different cleavage modules which can in turn be cleaved by different double strand-specific nucleases. It is likewise also possible for the probe nucleic acids to possess different labels, with the labels being fluorophores and/or parts of a binding pair.

An advantage of the method according to the invention is that the probe nucleic acids which are used in accordance with the invention contain different variables. On the one hand, it is possible to vary the target sequences and, as a result, it is possible to detect very different nucleic acid sequences in the sample to be investigated. On the other hand, the cleavage molecules can contain recognition sequences for a variety of restriction endonucleases. As a result, the probe nucleic acids can be digested with different restriction endonucleases either in parallel or sequentially. Finally, the probe nucleic acids can also possess different labels. The corresponding DNA array can then be configured in dependence on the nature of the task and several different treatment and evaluation steps can be carried out either in parallel or sequentially, with this making it possible to obtain a maximum amount of information.

The present invention also relates to a kit for detecting hybridizations with target nucleic acids, with it being possible to use the kit to carry out at least one hybridization with at least one probe nucleic acid which is used in accordance with the invention.

Techniques which are known from other nucleic acid detection methods can also be used within the context of the method according to the invention. In methods such as Northern blots, Southern blots or nuclease protection assays, the sequence-specific detection of target nucleic acids, which can be DNA or RNA, is effected by detecting the formation of hybrids (duplexes) composed of target nucleic acids and labeled probe nucleic acids, which can be DNA, RNA or PNA. In the case of DNA arrays, the unlabeled probe nucleic acid is bound to a solid matrix and is hybridized with labeled cDNA or cRNA (termed sample nucleic acids below). In general, hybridization events in nucleic acids are detected by detecting fluorescence, chemiluminescence, chemifluorescence or radioactivity [Sambrook, J. et al., “Molecular Cloning” 2001, 3rd Edition, Cold Spring Harbor Laboratory]. A number of fluorophores, such as fluorescein, lissamine, phycoerythrin, rhodamine (Perkin Elmer Cetus), Cy2, Cy3, Cy3.5, Cy5, Cy5.5, Cy7 and fluorx (Amersham), for example, can be used for fluorescence-labeling sample and/or probe nucleic acids [Kricka, L.: “Non isotopic DNA probe techniques”, 1992, Academic Press, San Diego]. In addition to the fluorophores which are listed here, it is also possible to use other fluorophores, which are not mentioned here, for labeling nucleic acids. These fluorophores include all fluorophores which can be linked covalently to nucleic acids and whose excitation and emission maxima are in the infrared range, in the visible range or in the UV range of the spectrum. If sample or probe nucleic acids are labeled with parts of a binding pair, such as biotin, digoxigenin or other haptens, the second part of the binding pair (streptavidin or antidigoxigenin Ab), which is conjugated with a detectable label, is incubated with the duplexes following hybridization. The detectable label of the second part of the binding pair can be a fluorophore or an enzyme (alkaline phosphatase, horseradish peroxidase, inter alia) which converts a substrate in conjunction with the emission of light (chemiluminescence or chemifluorescence) [“Fluorescent and Luminescent Probes for biological activity”, 1999, 2nd Edition, Mason, W. T. ed.].

Nucleic acids are labeled nonradioactively by carrying out the enzyme-catalyzed synthesis of DNA or RNA in the presence of nucleotide triphosphates whose nucleotide bases are linked covalently to fluorophores, parts of a binding pair (e.g. biotin, digoxigenin or other haptens) or reactive groups (NH₂ or SH). During the course of the synthesis, which is catalyzed by DNA or RNA polymerases (AMV reverse transcriptase, MMuLV reverse transcriptase, T7 RNA polymerase, T3 RNA polymerase, SP6 RNA polymerase, Taq polymerase, Klenow fragment, DNA polymerase and others, these modified nucleotide triphosphates are incorporated into the newly forming nucleic acid [Sambrook, J. et al., “Molecular Cloning” 2001, 3rd Edition, Cold Spring Harbor Laboratory]. The integrity of the mRNA which is used as a template for synthesizing the labeled sample nucleic acids is of crucial importance in this regard. The length of a probe nucleic acid, which is synthesized from RNA by means of reverse transcription and synthesized from DNA by means of PCR or in-vitro transcription, is, together with its base composition, of fundamental importance for the labeling efficiency. The shorter a nucleic acid to be labeled is, the few detectable group-modified nucleotide triphosphates (dNTPs or rNTPs) are incorporated during the synthesis. If an mRNA population is degraded by RNases, the sample nucleic acid which is synthesized from this RNA population is then, on the one hand, only very weakly labeled and, on the other hand, not representative of the transcription state which prevails in the cell type, tissue or organism to be investigated. A representative sample nucleic acid can only be synthesized from absolutely intact mRNA. Nucleotide triphosphates whose bases have been modified with the above-listed groups are incorporated by all known DNA and RNA polymerases, which catalyze the synthesis of DNA and RNA, respectively, with substantially lower efficiency than are unmodified nucleotide triphosphates [Molecular Dynamics Inc., “Fluorescent DNA-Labeling by PCR”, 1999, Molecular Dynamics Application Note # 62]. Thus, the yield of labeled probe nucleic acid is low as compared with radioactive systems and the loss of sample material resulting from the purification steps following the synthesis is very high.

For this reason, relatively large quantities of sample material, such as culture cells or tissue samples, from which the mRNA to be analyzed is isolated, have to be used for synthesizing nonradioactively labeled nucleic acids which are used, for example, for hybridizing DNA arrays. For this reason, DNA arrays can only be used to a restricted extent in areas, such as clinical diagnosis, in which the quantity of the sample material which is available for analysis is limited. Furthermore, nucleic acids have the property of attaching to the surface of dirt particles. For this reason, dirt particles can severely impair the detection, in particular on DNA arrays, of solid phase-bound unlabeled nucleic acids by hybridization with labeled nucleic acids, and in this way lead to falsely positive results.

In the field of biotechnology, and, in particular, in fields such as clinical diagnosis or industrial active compound research, which depend on automated methods for ensuring a high sample throughput, there is a need for methods for implementing expression analyses which enable the results of the measurement to be standardized without carrying out any elaborate calibration measurements. Systems for detecting hybridization events in nucleic acids which are in accordance with the prior art and which are used in the field of expression analysis depend greatly on the integrity of the mRNA which is to be investigated and which is used as the sample nucleic acid or as the template for synthesizing sample nucleic acids. On account, in particular, of the labeling of the non-solid phase-bound nucleic acids which are to be hybridized (sample nucleic acids) these systems are susceptible to erroneous interpretations which can falsify the entire measurement.

The method according to the invention can be used for expression analyses in which the sequence-specific hybridization of unlabeled target nucleic acid with solid phase-bound probes can be determined quantitatively regardless of the integrity of the sample nucleic acids. While the maximum number of different probe nucleic acids which can be detected on a surface is restricted by physical limits in the area of optics and microfluidics, the invention discloses a DNA array using a system in which, not restricted by the physical limits in the area of optics and microfluidics, the quantity of probe nucleic acids which can be detected on a defined area can be markedly increased as compared with systems which are in accordance with the prior art.

A preferred embodiment discloses a method for detecting hybridization events on solid phase-bound probe nucleic acids, which method, by using solid phase-bound labeled probes, makes it possible to standardize the measurement results, to use unlabeled sample or target nucleic acids and to simultaneously analyze the expression of different target nucleic acids on a defined spot. The solid phase-bound probe nucleic acids according to the invention, which can be DNA or DNA/PNA chimeras [Finn, P. J. et al.: “Synthesis and properties of DNA-PNA chimeric oligomers”, 1996, Nuc. Acids. Res., vol. 24 (17): Ratilainen, T. et al. : “Thermodynamics of sequence-specific binding of PNA to DNA”, 2000, Biochemistry, vol 39: van der Laan, A. C. et al.: “Optimization of the binding properties of PNA-(5′)-DNA-Chimerae”, 1998, Bioorg. Med. Chem. Lett., vol. 8], form, due to their sequences, intramolecular secondary structures which can be recognized and cleaved by double strand-specific endonucleases. The moiety of the probe nucleic acids according to the invention which is double-stranded as a result of the formation of the intramolecular secondary structure is in principle DNA in order to ensure accessibility for double strand-specific endonucleases.

As a result of hybridization of the solid phase-bound probe target nucleic acids according to the invention with nucleic acids to be detected, the intramolecular secondary structure, and thus the double-stranded region, is dissociated and can no longer be recognized and cleaved by double strand-specific endonucleases. Unhybridized, solid phase-bound probe nucleotides are cleaved enzymically. The labeled moiety of the probe nucleic acids according to the invention is separated off from the surface by the enzymic cleavage and diffuses into the surrounding medium and can, where appropriate, be washed out. After the unhybridized probe nucleic acids have been broken down enzymically, the fluorescence of the hybridized probe nucleic acids, which have not been truncated enzymically, is measured. The signal/background ratio of the method depends solely on the quality of the double strand-specific endonucleases employed and on the completeness with which the unhybridized probe nucleic acids are separated off and corresponds to that of a hybridization with radioactively labeled nucleic acids.

Since it is the solid phase-bound probe nucleic acids, rather than a particular sample or target nucleic acid, which are labeled, as many probe nucleic acids of differing sequence specificity can be immobilized on a defined area or spot on a surface as there are fluorophores which can be differentiated spectrally in regard to their excitation or emission maxima. The number of fluorophores which are linked covalently to the probe nucleic acid can be used to increase the sensitivity of the present method as desired.

In addition to the known fluorophores, it is also possible to use parts of a binding pair, such as digoxigenin, biotin or other haptens, for labeling the solid phase-bound probes. The molecules (immuno-globulins, streptavidin) which bind the different haptens specifically are covalently linked to enzymes of differing substrate specificities. These enzymes can be alkaline phosphatase, peroxidase, acid phosphatase and others. It is thus possible to immobilize as many probe nucleic acids of differing sequence specificity on a defined area or spot on a surface as desired depending on the number of different haptens which are available and on the number of different enzyme conjugates which are available.

The sample or target nucleic acids which are used for hybridizing with the immobilized probe nucleic acids can be unlabeled DNA, cDNA, cRNA or mRNA. In contrast to conventional systems, it is only the portion of the target or sample nucleic acid which is complementary to the detection module of the probe nucleic acid which has to be used for the hybridization since, in the present system, the probe nucleic acid is labeled. Another advantage of the described system is that the sensitivity of the detection does not depend on the efficiency with which the sample nucleic acids are labeled but, instead, solely on the labeling of the probe nucleic acid. This latter can, however, be determined very much more precisely.

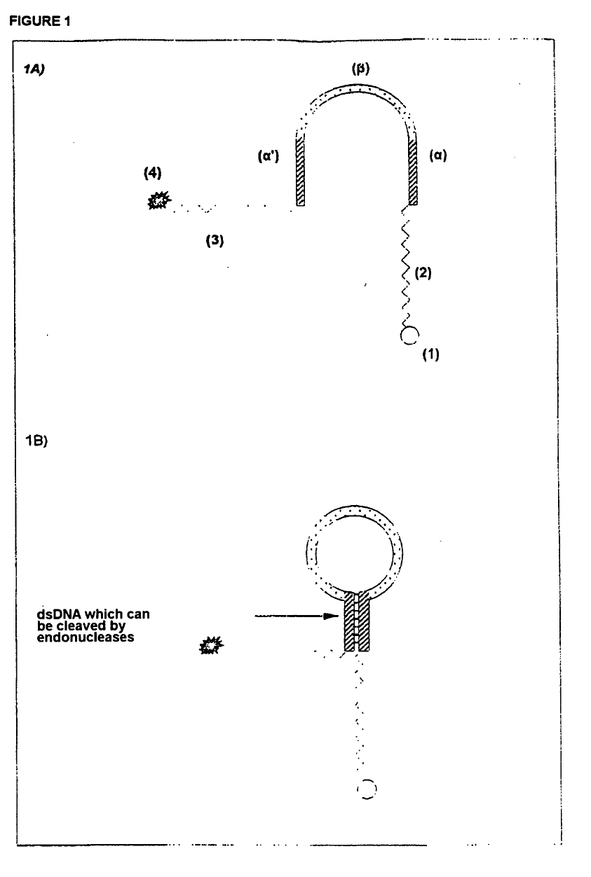

A probe nucleic acid according to the invention is represented diagrammatically in FIG. 1A. A typical probe nucleic acid possesses the following components:
at least one functional group (1), such as an amino group (NH2) or thiol group (SH) or a part of a binding pair, such as biotin or digoxigenin, for binding to a solid phase.
A spacer module (2), which is preferably more than 11 C2 bonds in length and one of whose ends is linked covalently to the functional group.
A sequence segment α (recognition and cleavage module) which is preferably 5-12 nucleotides in length, which consists of DNA and whose 3′ end is linked covalently to the end of the spacer module which is linked to the solid phase. Sequence segment α contains the recognition sequence for a restriction endonuclease.
A sequence segment β (detection module) which is preferably from 12 to 30 nucleotides in length, whose 3′ end is linked covalently to the 5′ end of sequence segment a (recognition and cleavage module) and which can be DNA, RNA or PNA. Sequence segment β constitutes the portion of the molecule which is able, under suitable reaction conditions, to form, as probe, a heteroduplex with a target nucleic acid to be detected. The nucleotides and/or the sugar phosphate backbone or pseudopeptide backbone of sequence segment β can be covalently linked to fluorophores.
A sequence segment α′ (recognition and cleavage module) which consists of DNA and whose 3′ end is covalently linked to the 5′ end of sequence segment β. The sequence of segment α′ is complementary to that of segment α.
A spacer module (3) which is preferably more than 11 C2 bonds in length and which is linked covalently to the 5′ end of sequence segment α′.
Where appropriate, a branching module [Newcome, G. R. et al.: “Dendritic Molecules: Concepts, Synthesis, Perspectives”, 1996, VCH Publishers] (not depicted) which is linked covalently to the end of spacer module (3) which is not linked to the 5′ end of sequence segment α′. Up to n additional branching modules can be bonded to the individual ends of this branching module, thereby giving rise to a maximum number of 3ⁿ ends, with
a fluorophore (4), or parts of a binding pair (biotin or digoxigenin), being bonded to each end.

The probe is bound to a solid matrix by way of element (1). This solid surface can, inter alia, be a plane surface, which can also be convex or concave, a fiber or a microparticle or nanoparticle composed of inorganic or organic material. The probe nucleic acids according to the invention which are bound to such a solid matrix are termed a DNA array below. Sequence segments α and α′ are complementary to each other and are able, under suitable conditions, to form a double-stranded region, i.e. the duplex α-α′ (see FIG. 1B). In the case of this molecule, the formation of a hairpin structure by means of intramolecular duplex formation is favored thermodynamically over the single-stranded conformation. Thus, at a temperature which is lower than the equilibrium melting temperature Tₘ of the sequence segment α or α′, the molecule is exclusively present as a hairpin structure containing the intramolecular duplex α-α′, which can be cleaved by restriction endonucleases in a sequence-specific manner.

Following cleavage by a restriction endonuclease, the elements (1) and (2) and, where appropriate, a few nucleotides of the element (α) remain bound to the solid phase. Labeled elements of the probe diffuse into the surrounding medium and are removed, where appropriate, by washing.

As a result of hybridizing with a target nucleic acid, which can be RNA or DNA and whose sequence is complementary to the sequence of sequence segment β, the probe is partially present as a duplex with the sample nucleic acid. In this case, the sequence segments α and α′ are present as single strands and cannot be cleaved by restriction endonucleases or other double strand-specific nucleases. In order to ensure sufficient stability of the heteroduplex composed of sample nucleic acid and sequence segment β as compared with the intramolecular duplex α-α′, the equilibrium melting temperature (Tₘ(β) of sequence segment β must be higher than that of sequence segment α or α′; that is Tₘ(β)>Tₘ(α) [Bonnet, G. et al., “Thermodynamic basis of the enhanced specificity of structured DNA probes”, 1999, Proc. Natl. Acad. Sci., vol. 96]. Ideally, the equilibrium melting temperature Tₘ(β) of the hetero-duplex formed from sample nucleic acid and sequence segment β is between 10° C. and 25° C. higher than the equilibrium melting temperature Tₘ(α) of the intramolecular duplex α-α′. A probe nucleic acid which is able to form a secondary structure has a higher sequence specificity with the same target nucleic acid than does a linear probe nucleic acid. The difference in the equilibrium melting temperature, i.e. ΔTₘ, between a probe/target nucleic acid duplex which does not contain any base mispairing and a probe/target nucleic acid duplex which contains one base mispairing is about twice as high in the case of probe nucleic acids which are able to form a secondary structure as in the case of linear probe nucleic acids [Bonnet, G. et al., “Thermodynamic basis of the enhanced specificity of structured DNA probes”, 1999, Proc. Natl. Acad. Sci., vol. 96].

The method according to the invention can preferably be employed for multiplex analyses. In this case, n different probe nucleic acids which differ from each other in sequence segment β, in the excitation and emission spectra of the fluorophores [Vet, J. A. M. et al., “Multiplex detection of four pathogenic retroviruses using molecular beacons”, 1999, Proc. Natl. Acad. Sci., vol. 96; Marras, S. A. E. et al., “Multiplex detection of single-nucleotide variations using molecular beacons”, 1999, Genetic Analysis: Biomolecular Engineering, vol. 14] and, where appropriate, with regard to the recognition sequences for restriction endonucleases present in sequence segments α and α′ are immobilized on the same area. Printing methods are used to deposit and immobilize an aqueous solution containing equimolar quantities of these n different probe nucleic acids on a solid surface [Cheung, V. G. et al., “Making and reading microarrays”, Nature Genetics, vol. 21, January 1999; Bowtell, D. D. L., “Options available—from start to finish—for obtaining expression data by microarray”, Nature Genetics, vol. 21, January 1999]. This area can be a spot on a DNA array or the surface of a microparticle or nanoparticle.

In this way, it is possible to simultaneously analyze the hybridization of n different target nucleic acids of different sequence with n different probe nucleic acids whose sequence segments α and α′ contain the same recognition sequence for a restriction endonuclease. If the sequence segments α and α′ of the probe nucleic acids contain n different recognition sequences for n different restriction endonucleases, it is then possible to simultaneously, or preferably serially, analyze the hybridization of n different target nucleic acids of differing sequence with n different probe nucleic acids.

The method according to the invention can be carried out in the following way for the purpose of implementing a multiplex analysis with regard to the hybridization of the probe nucleic acid with target or sample nucleic acids:

a DNA array composed of one or more probe nucleic acids according to the invention which is/are bound to a solid matrix is brought into contact with unlabeled sample nucleic acid, which can be RNA or DNA, under the preferred conditions which are cited below. The DNA array is incubated, at 45° C. for 10-20 minutes and in accordance with the surface to be hybridized, with 20 μl-200 μl of a suitable hybridization buffer.

0.1 μg-50 μg of unlabeled sample nucleic acid is/are taken up in 300 μl of suitable hybridization solution and, prior to the hybridization with the DNA array, this solution is heated at about 99° C. for 5 minutes and then cooled down to about 45° C. for 5 minutes. The hybridization buffer is removed from the DNA array and replaced with the hybridization solution containing the sample nucleic acids. The DNA array is incubated with the sample nucleic acids at 45° C.-60° C. for 16 hours. After the sample nucleic acid solution has been removed, the DNA array is washed with washing buffers of differing ionic strength in each case at 50° C.-65° C. The choice of the suitable hybridization and washing conditions depends on the nature of the sample nucleic acid (DNA or RNA) and its length and also on the nature (DNA, RNA or PNA) and length of the immobilized probe nucleic acids [Anderson, M. L. M.: “Nucleic acid Hybridization”, 1998, Springer-Verlag Telos; Schena, M.: “DNA-Microarrays: A practical approach”, 1999, Oxford University Press].

The cleavage of the unhybridized probe nucleic acids by restriction endonucleases in the region of the α-α′ duplex is preferably effected at 25° C.-37° C. under the reaction conditions recommended by the manufacturer. The activity of the restriction endonucleases can be increased up to 34-fold by adding lipids to the reaction mixture [Kinnunen et al., “Materials and methods for digestion of DNA or RNA using restriction endonucleases”, U.S. Pat. No. 5,879,950]. After the washing steps, the DNA array is incubated, at 25° C.-37° C. for 20-60 minutes, and in accordance with the surface to be hybridized, with 20 μl-200 μl of the reaction buffer recommended by the manufacturer, with the buffer containing 0.5 U-5 U of the restriction endonuclease which cleaves the probe nucleic acid in the region of the α-α′ duplex. Cleaved-off probe nucleic acids, labels and restriction endonucleases are removed from the surface of the DNA array by washing, at room temperature, with a 1× TE buffer.

The signals and data which are obtained by means of the method according to the invention are preferably detected and analyzed in the following way:

Before using printing methods [Cheung, V. G. et al., “Making and reading microarrays”, Nature Genetics, vol. 21, January 1999; Bowtell, D. D. L., “Options available—from start to finish—for obtaining expression data by microarray”, Nature Genetics, vol. 21, January 1999] to transfer the labeled probes to, and immobilize them on, a solid surface, the degree of labeling of each probe nucleic acid is determined. The nucleic acid concentration is determined by measuring the absorption of an aqueous nucleic acid solution at a wavelength of 260 nm and using Lambert-Beer's law (A₂₆₀=ε×d×c, where A₂₆₀ is the absorption at λ=260 nm, ε is the molar extinction coefficient (cm⁻¹M⁻¹] of the nucleic acid, with this coefficient depending on the base sequence and length of the nucleic acid to be investigated, d is the path length of the cuvette employed and c is the concentration [M] of the nucleic acid).

The concentration of the fluorophores which are conjugated with the probe nucleic acid is determined by measuring the absorption of an aqueous solution of the labeled probe nucleic acids at a wavelength which corresponds to the absorption maximum of the fluorophores (λₘₐₓ) and using Lambert-Beer's law. In order to be able to determine precisely the concentration of a nucleic acid which is conjugated with fluorophores, account must be taken of the fact that most fluorophores absorb light having a wavelength of 260 nm. The contribution provided by the fluorophores, which is the product of the absorption of the fluorophores at the wavelength which corresponds to their absorption maximum (λₘₐₓ) and a correction factor CF₂₆₀, is subtracted from the total absorption at 260 nm, i.e. (A_{nucleic acid}=A₂₆₀−(A_{λmax}×CF₂₆₀)). The molar extinction coefficients of different fluorophores, and the correction factor for the absorption at 260 nm (CF₂₆₀), can be obtained from the manufacturers of these fluorophores (Molecular Probes, BioRad and others).

The ratio of the concentration of the fluorophores which are conjugated with the probe nucleic acid to the. concentration of the probe nucleic acid is equal to the quantity of the fluorophores which are conjugated with the probe nucleic acid. In order to determine the specific fluorescence of the labeled probe nucleic acids, the fluorescence of a specified quantity of these probe nucleic acids is determined. The specific fluorescence can then be used to precisely determine, prior to a hybridization with target or sample nucleic acid, the number of probe nucleic acid molecules which are bound to a solid matrix. In this way, variations in the quantity of probe nucleic acids immobilized, which variations affect the hybridization with target or sample nucleic acids, can be taken into account and corrected by calculation. It is therefore possible to standardize measurements which are carried out on the basis of hybridizing target or sample nucleic acids with probe nucleic acids according to the invention.

The fluorescence emission of each sample spot in the DNA array is preferably determined by means of confocal laser scanning microscopy. Instruments for determining fluorescence emission on small areas are offered for sale by a number of manufacturers and are a laboratory standard in the field of biotechnology [Cheung, V. G. et al., “Making and reading microarrays”, Nature Genetics, vol. 21, January 1999; Bowtell, D. D. L., “Options available—from start to finish—for obtaining expression data by microarray”, Nature Genetics, vol. 21, January 1999]. In order to standardize the measurement results, the fluorescence of the immobilized probe nucleic acids is determined prior to a hybridization. If a probe nucleic acid which is of a particular sequence specificity, and which is labeled with a particular fluorophore, is immobilized on each sample spot of the array, the fluorescence is excited by light of a wavelength which corresponds to the absorption maximum (λ_{abs.max}) of this fluorophore and is detected at a wavelength which corresponds to the emission maximum (λ_{em.max}). If n different probe nucleic acids, which are of differing sequence specificity and which are labeled with n different, spectrally distinguishable fluorophores, are immobilized on each sample spot of the DNA array, the fluorescence of the n different fluorophores is excited by light of a wavelength which corresponds to the absorption maximum (λ_{abs.max}) of these fluorophores and is detected at a wavelength which corresponds to the emission maximum (λ_{em.max}). Depending on the instrument employed, the fluorescence of different fluorophores can be determined simultaneously or consecutively.

The present invention is clarified by means of the following examples:

### EXAMPLE 1

Doubly modified oligodeoxynucleotides, whose 5′ and 3′ ends are in each case covalently linked, by way of a C22 spacer, to fluorescein isothiocyanate (FITC) and, respectively, an amino group (NH₂) (sequence A: FITC-^{5′}gcccgcgcAATAGGGATGGCTCAACAgcgcgggc₃-(C22)NH₂ and B: FITC-^{5′}gcccgcgcTTAGAGTGCAAAATGAAAGCGCCgcgcgggc₃-(C22)NH₂) were taken up in 100 μl of coupling buffer (500 mM Na₂HPO₄, pH 8.5, 1 mM EDTA) (concentration: 500 pmol/ml). By incubating in each case 100 μl of the oligonucleotide solution at RT (room temperature) for 30 min in the wells of a microtiter plate (Thermowell M PCR plate, Corning* Surface Technologies), the oligonucleotides were bonded covalently to the surface of the wells, as shown in Table 1:

TABLE 1
Positions of the sample nucleic acids A and B on the microtiter plate

| | 1 | 2 | 3 | 4 | 5 | 6 | 7 | 8 | 9 | 10 | 11 | 12 |
|---|---|---|---|---|---|---|---|---|---|---|---|---|
| A | A | — | — | — | A | — | — | — | A | — | — | — |
| B | — | — | B | — | — | — | B | — | — | — | B | — |
| C | A | — | — | — | A | — | — | — | A | — | — | — |
| D | — | — | B | — | — | — | B | — | — | — | B | — |
| E | A | — | — | — | A | — | — | — | A | — | — | — |
| F | — | — | B | — | — | — | B | — | — | — | B | — |
| G | A | — | — | — | A | — | — | — | A | — | — | — |
| H | — | — | B | — | — | — | B | — | — | — | B | — |

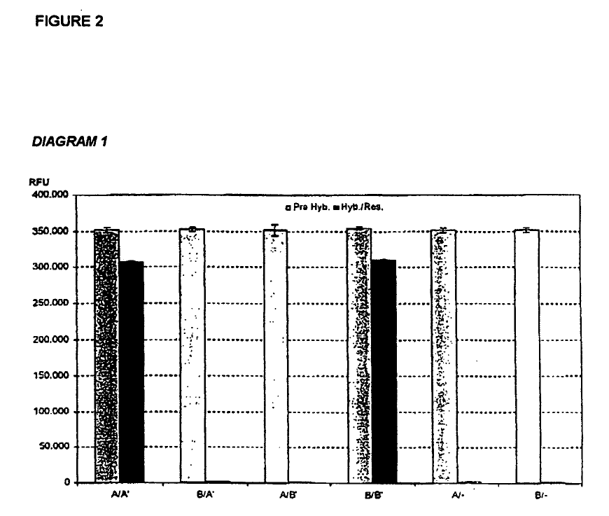

The wells of the microtiter plate were then washed 5× with 200 μl of 10 mM Tris, pH 8.0, 150 mM NaCl. The fluorescence intensity of the oligonucleotides which were bound in the wells of the microtiter plate was determined in a spectrofluorimeter (Molecular Devices: Spectramax Gemini XS) at the excitation wavelength λ_{abs.max}=490 nm and the emission wavelength λ_{em.max}=520 nm. The data are listed in table 3 and depicted in FIG. 2 (diagram 1):

TABLE 3
Fluorescence intensity × 1000 of the oligonucleotides which were
covalently bound in the wells of a microtiter plate

| | 1 | 2 | 3 | 4 | 5 | 6 | 7 | 8 | 9 | 10 | 11 | 12 |
|---|---|---|---|---|---|---|---|---|---|---|---|---|
| A | 352 | 0 | 0 | 0 | 348 | 0 | 0 | 0 | 351 | 0 | 0 | 0 |
| B | 0 | 0 | 351 | 0 | 0 | 0 | 356 | 0 | 0 | 0 | 351 | 0 |
| C | 356 | 0 | 0 | 0 | 355 | 0 | 0 | 0 | 348 | 0 | 0 | 0 |
| D | 0 | 0 | 350 | 0 | 0 | 0 | 353 | 0 | 0 | 0 | 349 | 0 |
| E | 348 | 0 | 0 | 0 | 350 | 0 | 0 | 0 | 356 | 0 | 0 | 0 |
| F | 0 | 0 | 356 | 0 | 0 | 0 | 354 | 0 | 0 | 0 | 352 | 0 |
| G | 351 | 0 | 0 | 0 | 352 | 0 | 0 | 0 | 352 | 0 | 0 | 0 |
| H | 0 | 0 | 353 | 0 | 0 | 0 | 351 | 0 | 0 | 0 | 356 | 0 |

The wells of the microtiter plate were prehybridized, at 60° C. for 4 hours, with in each case 125 μl of hybridization solution which contained 0.1 mg of sheared salmon sperm DNA/ml (Gibco BRL/Life Technologies); 0.5 mg of acetylated BSA/ml (Gibco BRL/Life Technologies); 1× MES (100 mM MES, 1.0M NaCl, 20 mM EDTA, 0.01% Tween 20).

After the prehybridization solution had been removed, 125 μl of hybridization solution, which contained 1 nmol of sample RNA oligonucleotides (sequences A′=^{5′}UGUUGAGCCAUCCCUAUU_{3′} and, respectively, B′=^{5′}GGCGCUUUCAUUUUGCACUCUAA_{3′}) in 0.1 mg of sheared salmon sperm DNA/ml (Gibco BRL/Life Technologies); 0.5 mg of acetylated BSA/ml (Gibco BRL/Life Technologies); 1× MES (100 mM MES, 1.0M NaCl, 20 mM EDTA, 0.01% Tween 20), were added to the wells of the microtiter plate. The set-up is shown in table 2:

TABLE 2
Sectors of the microtiter plate designated A′, B′ and - were
hybridized with the sample nucleic acids A′ and B′ and with
prehybridization solution, respectively.

| | 1 | 2 | 3 | 4 | 5 | 6 | 7 | 8 | 9 | 10 | 11 | 12 |
|---|---|---|---|---|---|---|---|---|---|---|---|---|
| A | A′ | A′ | A′ | A′ | B′ | B′ | B′ | B′ | — | — | — | — |
| B | A′ | A′ | A′ | A′ | B′ | B′ | B′ | B′ | — | — | — | — |
| C | A′ | A′ | A′ | A′ | B′ | B′ | B′ | B′ | — | — | — | — |
| D | A′ | A′ | A′ | A′ | B′ | B′ | B′ | B′ | — | — | — | — |
| E | A′ | A′ | A′ | A′ | B′ | B′ | B′ | B′ | — | — | — | — |
| F | A′ | A′ | A′ | A′ | B′ | B′ | B′ | B′ | — | — | — | — |
| G | A′ | A′ | A′ | A′ | B′ | B′ | B′ | B′ | — | — | — | — |
| H | A′ | A′ | A′ | A′ | B′ | B′ | B′ | B′ | — | — | — | — |

The set-up is heated at 95° C. for 20 min, cooled down to 60° C. over the course of one hour and hybridized at 60° C. for 16 hours.

After the sample nucleic acid solution had been removed, the wells of the microtiter plate were washed ten times, at 25° C. for 5 minutes, with “non-stringent” washing buffer (6× SSPE; 0.01% Tween 20) and then washed 5 times, at 55° C. for 5 minutes, with “stringent” washing buffer (100 mM MES; 0.1 M NaCl; 0.01% Tween 20).

After the washing, the wells of the microtiter plate were equilibrated, at 37° C. for ten minutes, with 150 μl of 1× reaction buffer (NEB Buffer 3) and then incubated, at 37° C. for one hour, with in each case 100 μl of 1× reaction buffer which contained 2 units of the restriction endonuclease Acil (New England Biolabs). The reaction was stopped by adding ⅕ of the volume of stop solution (0.5% w/v SDS, 50 mM EDTA) and heating the microtiter plate to 75° C. After that, the wells of the microtiter plate were washed 6 times, at room temperature, with in each case 150 μl of 1× TE buffer (10 mM Tris, 1 mM EDTA, pH=8.0). All the incubations which were not carried out at room temperature were carried out in a Biometra UNO™ thermoblock with lid heating. The fluorescence intensity in the individual wells of the microtiter plate was determined in a spectrofluorimeter (Molecular Devices: Spectramax Gemini XS) at the excitation wavelength λ_{abs.max}=490 nm and the emission wavelength λ_{em.max}=520 nm. The data are shown in table 4 and depicted in diagram 1 (FIG. 2):

TABLE 4
Fluorescence intensity × 1000, following hybridization and
restriction digestion, of the oligonucleotides which were bound
covalently in the wells of a microtiter plate

| | 1 | 2 | 3 | 4 | 5 | 6 | 7 | 8 | 9 | 10 | 11 | 12 |
|---|---|---|---|---|---|---|---|---|---|---|---|---|
| A | 308 | 0 | 0 | 0 | 2.06 | 0 | 0 | 0 | 2.11 | 0 | 0 | 0 |
| B | 0 | 0 | 2.11 | 0 | 0 | 0 | 309 | 0 | 0 | 0 | 2.11 | 0 |
| C | 309 | 0 | 0 | 0 | 2.16 | 0 | 0 | 0 | 2.01 | 0 | 0 | 0 |
| D | 0 | 0 | 2.14 | 0 | 0 | 0 | 308 | 0 | 0 | 0 | 2.1 | 0 |
| E | 306 | 0 | 0 | 0 | 2.01 | 0 | 0 | 0 | 2.14 | 0 | 0 | 0 |
| F | 0 | 0 | 2.14 | 0 | 0 | 0 | 354 | 0 | 0 | 0 | 2.12 | 0 |
| G | 307 | 0 | 0 | 0 | 2.15 | 0 | 0 | 0 | 2.12 | 0 | 0 | 0 |
| H | 0 | 0 | 2.12 | 0 | 0 | 0 | 351 | 0 | 0 | 0 | 2.13 | 0 |

The signal which was detectable in the region of the wells 2A-2H, 4A-4H, 6A-6H, 8A-8H, 10A-10H, 12A-12H, 1B, 1D, 1F, 1H, 3A, 3C, 3E, 3G, 5B, 5D, 5F, 5H, 7A, 7C, 7E, 7G, 9B, 9D, 9F, 9H, 11B, 11D, 11E and 11G corresponds to the background fluorescence of the system and is subtracted from the signals which were detected in the region of the sample nucleic acids A and B.

### EXAMPLE 2

The fluorescein isothiocyanate (FITC)-labeled sample nucleic acids of the sequence

| | |
|---|---|
| A: | FITC-^{5′}gcccgcgcAATAGGGATGGCTCAACAgcgcgggc₃, |
| B: | FITC-^{5′}gcccgcgcTTAGAGTGCAAAATGAAAGCGCCgcgcgggc₃ |
| and | |
| C: | FITC-^{5′}gcccgcgcGTTTTTTTTTTTTGGTTTTTTTTTTTC-gcgc |
| gggc_{3′} | |

(control: determination of the background fluorescence), which are immobilized at different sample spots on a solid matrix, are hybridized, at 55° C. for 16 hours, with 5 μg of sample RNA, which was isolated from K562 cells, in 25 pM control RNA, 0.1 mg of sheared salmon sperm DNA/ml (Gibco BRL/Life Technologies); 0.5 mg of acetylated BSA/ml (Gibco BRL/Life Technologies); 1× MES (100 mM MES, 1.0M NaCl, 20 mM EDTA, 0.01% Tween 20). After the sample nucleic acid solution has been removed, the DNA array is washed ten times, at 25° C. for 5 minutes, with “non-stringent” washing buffer (6× SSPE; 0.01% Tween 20) and then five times, at 50° C. for 5 minutes, with “stringent” washing buffer (100 mM MES; 0.1M NaCl; 0.01% Tween 20).

After the washing, the DNA array is equilibrated with 1× reaction buffer at 37° C. for 10 minutes. The DNA array is then incubated, at 37° C. for 1 hour, in 100 μl of 1× reaction buffer which contains 2 units of the restriction endonuclease Acil. The reaction is stopped by adding ⅕ of the volume of stop solution (0.5% w/v SDS, 50 mM EDTA) and by heating the DNA array to 75° C. The DNA array is then washed 4-8 times, at room temperature, in 1× TE (10 mM Tris, 1 mM EDTA, pH=8.0). The fluorescence of the hybridized probes, which have remained on the DNA array, is excited by light of a wavelength λ_{abs.max}=490 nm and the emitted light is detected at a wavelength λem.max=520 nm. The signal which can be detected in the region of the sample nucleic acid C corresponds to the background fluorescence of the system and is subtracted from the signals which are detected in the region of the sample nucleic acids A and B.

The sample nucleic acids of the sequence

| | |
|---|---|
| A: | (FITC)-^{5′}gcccgcgcAATAGGGATGGCTCAACA- |
| | gcgcgggc_{3′}, |
| B: | (Cascade Blue)-^{5′}gcccgcgcTTAGAGTGCAAAATGAAAGCGC |
| | Cgcgcgggc₃ |
| and | |
| C: | (BODIPY TR14)-^{5′}gcccgcgcTTTCTCTACCTCCTCACATTGTG |
| | gcgcgggc_{3′}, |

which are labeled with the fluorophores FITC (λ_{Abs.max}=490 nm, λ_{Em.max}=520 nm), Cascade Blue (λ_{Abs.max}=400 nm, λ_{Em.max}=420 nm) and BODIPY TR14 (λ_{Abs.max}=595 nm, λ_{Em.max}=625 nm), are immobilized jointly on a defined area A (sample spot A). The probe nucleic acids D: (FITC)-^{5′}gcccgcgcGTTTTTTTTTTTTGGTTTTTTTTTTTC-gcgcgggc_{3′}, E: (Cascade Blue) -^{5′}gcccgcgcGTTTTTTTTTTTTGGTTTTTTTTTTTC-gcgcgggc_{3′} and F: (BODIPY TR14)-^{5′}gcccgcgcGTTTTTTTTTTTTGGTTTTTTTTTTTC-gcgcgggc_{3′}, which are used as a control for determining the background fluorescence, are immobilized jointly on another defined area B (sample spot D). The DNA array is hybridized, at 55° C. for 16 hours, with 0.1 μg of sample RNA (cRNA), which contains the sequences D′=^{5′}UGUUGAGCCAUCCCUAUU_{3′}, E′=^{5′}GGCGCUUUCAUUUUGCACUCUAA_{3′} and F′=^{5′}CACAAUGUGAGGAGGUAGAGAAA_{3′}, in 25 pM control RNA, 0.1 mg of sheared salmon sperm DNA/ml (Gibco BRL/Life Technologies); 0.5 mg of acetylated BSA/ml (Gibco BRL/Life Technologies): 1× MES (100 mM MES, 1.0M NaCl, 20 mM EDTA, 0.01% Tween 20). After the sample nucleic acid solution has been removed, the DNA array is washed ten times, at 25° C. for 5 minutes, with “non-stringent” washing buffer (6× SSPE; 0.01% Tween 20) and then five times, at 50° C. for 5 minutes, with “stringent” washing buffer (100 mM MES; 0.1M NaCl; 0.01% Tween 20).

After the washing, the DNA array is equilibrated with 1× reaction buffer at 37° C. for 10 minutes. The DNA array is then incubated, at 37° C. for 1 hour, in 100 μl of 1× reaction buffer which contains 2 units of the restriction endonuclease Acil. The reaction is stopped by adding ⅕ of the volume of stop solution (0.5% w/v SDS, 50 mM EDTA) and by heating the DNA array to 75° C. The DNA array is then washed 4-8 times, at room temperature, in 1× TE (10 mM Tris, 1 mM EDTA, pH=8.0). The fluorescence of the hybridized probes, which have remained on the DNA array and which are labeled with the fluorophorees FITC, Cascade Blue or BODIPY TR14, is excited by light of the wavelengths λ_{Abs.max}=490 nm, λ_{Abs.max}=400 nm and, respectively, λ_{Abs.max}=595 nm, and the light which is emitted at a wavelength of λ_{Em.max}=520 nm, λ_{Em.max}=420 nm and, respectively, λ_{Em.max}=625 nm is detected. The signals which can be detected in the region of the sample spot B correspond to the background fluorescence of the system and are subtracted from the signals which are detected in the region of the sample spot A.

1-7. (canceled)

8. A method for detecting the presence of and/or quantifying a target polynucleotide in a sample comprising:
(a) contacting a sample containing a target polynucleotide under hybridizing conditions with an array comprising at least one probe attached to a surface of the array, the probe comprising:
(i) a sequence segment (β) having a 3′ end and a 5′ end, which sequence segment is complementary to at least a portion of the target polynucleotide,
(ii) a first sequence segment (α) covalently attached to one of the two ends of the sequence segment (β),
(iii) a second sequence segment (α′), which is at least partially complementary to the first sequence segment (′), and is covalently attached to the other end of the sequence segment (β),
(iv) a means for attaching the probe to a surface of the array, the means being covalently linked to a free end of one of the first or second sequence segments (α) or (α′),
(v) a label attached to the end of the first or second sequence segments (α) or (α′), which is not linked to the means for attaching the probe,
the first and second sequence segments (α) and (α′), respectively forming a duplex cleavage module containing a recognition site for a restriction endonuclease when the sequence segment (β) does not hybridize with the target polynucleotide;
(b) treating the array with a restriction endonuclease, which cleaves at the recognition site formed when an (α)-(α′) duplex cleavage module is formed;
(c) detecting and/or quantifying the presence of the target polynucleotide in the sample by the amount of label bound to the array.

9. The method of claim 8 wherein the array comprises a plurality of probes, each probe having a distinct sequence segment (β), which is complementary to a distinct target polynucleotide.

10. The method of claim 8 wherein the array comprises a plurality of probes, each probe forming a distinct duplex cleavage module containing a recognition site for a distinct restriction endonuclease.

11. The method of claim 8 wherein the array comprises a plurality of probes, each probe having a different label.

12. The method according to claim 11 wherein the label is selected from the group consisting of fluorophores, members of a binding pair, and combinations thereof.

13. A kit for detecting and/or quantifying a target polynucleotide sequence in a sample comprising:
(α) a probe comprising:
(i) a sequence segment (β) having a 3′ end and a 5′ end, which sequence segment is complementary to at least a portion of the target polynucleotide,
(ii) a first sequence segment (α) covalently attached to one of the two ends of the sequence segment (β),
(iii) a second sequence segment (α′), which is at least partially complementary to the first sequence segment (α), and is covalently attached to the other end of the sequence segment (β),
(iv) a means for attaching the probe to an array, the means being covalently linked to a free end of one of the first or second sequence segments (α) or (α′),
(v) a label attached to the end of the first or second sequence segments (α) or (α′), which is not linked to the means for attaching the probe,
the first and second sequence segments (α) and (α′), respectively forming a duplex cleavage module containing a recognition site for a restriction endonuclease when the sequence segment (β) does not hybridize with the target polynucleotide; and
(b) reagents necessary for implementing the detection/quantification of the target polynucleotide.

14. An array comprising:
(a) a solid phase; and
(b) a probe covalently attached to a surface of the solid phase comprising:
(i) a sequence segment (β) having a 3′ end and a 5′ end, which sequence segment is complementary to at least a portion of the target polynucleotide,
(ii) a first sequence segment (α) covalently attached to one of the two ends of the sequence segment (β),
(iii) a second sequence segment (α′), which is at least partially complementary to the first sequence segment (α), and is covalently attached to the other end of the sequence segment (β),
(iv) a means for covalently attaching the probe to a surface of the solid phase, the means being covalently linked to a free end of one of the first or second sequence segments (α) or (α′),
(v) a label attached to the end of the first or second sequence segments (α) or (α′), which is not linked to the means for attaching the probe,
the first and second sequence segments (α) and (α′), respectively forming a duplex cleavage module containing a recognition site for a restriction endonuclease when the sequence segment (β) does not hybridize with the target polynucleotide.


SEQUENCE LISTING (7 sequences)

SEQ ID NO: 1 - DNA, 34 bp, Artificial
  Synthetic oligonucleotide.
gcccgcgcaatagggatggctcaacagcgcgggc

SEQ ID NO: 2 - DNA, 39 bp, Artificial
  Synthetic oligonucleotide.
gcccgcgcttagagtgcaaaatgaaagcgccgcgcgggc

SEQ ID NO: 3 - RNA, 18 nt, Artificial
  Synthetic oligonucleotide.
uguugagccaucccuauu

SEQ ID NO: 4 - RNA, 23 nt, Artificial
  Synthetic oligonucleotide.
ggcgcuuucauuuugcacucuaa

SEQ ID NO: 5 - DNA, 43 bp, Artificial
  Synthetic oligonucleotide.
gcccgcgcgttttttttttttggtttttttttttcgcgcgggc

SEQ ID NO: 6 - DNA, 39 bp, Artificial
  Synthetic oligonucleotide.
gcccgcgctttctctacctcctcacattgtggcgcgggc

SEQ ID NO: 7 - RNA, 23 nt, Artificial
  Synthetic oligonucleotide.
cacaaugugaggagguagagaaa